(19) United States
(12) Patent Application Publication
(10) Pub. No.: US 2025/0073093 A1
(43) Pub. Date: Mar. 6, 2025

(54) LOW CALIPER FEMININE HYGIENE PAD WITH TEXTURED TOPSHEET

(71) Applicant: The Procter & Gamble Company, Cincinnati, OH (US)

(72) Inventors: Amit Kumar KAUSHIK, West Chester, OH (US); Kelyn Anne ARORA, Cincinnati, OH (US); Andrea PHANTU, Cincinnati, OH (US); Digvijay RAWAT, Cincinnati, OH (US)

(21) Appl. No.: 18/241,460

(22) Filed: Sep. 1, 2023

(51) Int. Cl. A61F 13/472; A61F 13/15; A61F 13/511; A61F 13/514

(52) U.S. Cl. CPC A61F 13/472; A61F 13/15707; A61F 13/51104; A61F 13/514; A61F 2013/5113; A61F 2013/51165; A61F 2013/51182; A61F 2013/51366

(57) ABSTRACT

An absorbent article having a liquid permeable topsheet, a liquid impermeable backsheet, and an absorbent layer enveloped therebetween is disclosed. The topsheet comprises a fibrous nonwoven material and has an upper topsheet surface and a lower topsheet surface. The lower topsheet surface exhibits a lower topsheet surface Sdc less than 0.350 mm, and the upper topsheet surface exhibits an upper topsheet surface Sdc that is greater than the lower topsheet surface Sdc. Additionally, or alternatively, the lower topsheet surface exhibits a lower topsheet surface Sdr % less than 0.80; and the upper topsheet surface exhibits an upper topsheet surface Sdr % that is greater than the lower topsheet surface Sdr %. The absorbent layer comprises a layer of open-cell foam having an upper foam surface that is in direct facing contact with the lower topsheet surface.

### BACKGROUND

Some segments of the consumer market for absorbent hygiene products such as feminine hygiene pads prefer products with topsheets formed of nonwoven web materials. Alternative topsheet materials include apertured films, but these may not be preferred by some market segments for reasons relating to feel against the skin.

Topsheet nonwoven materials may be imparted with texture via embossing techniques. It is believed that consumers/users of absorbent hygiene products, such as feminine hygiene pads, appreciate visible macroscopic three-dimensional (3D) texture in the topsheet, for several reasons. These include a perception that a textured surface is more comfortable, more effective at trapping and preventing outward planar migration of discharged fluid (possibly off the wearer-facing surface of the pad), and aesthetic appeal. In certain circumstances, the perception of efficacy can have a factual basis—an appropriately textured topsheet combined with other suitable product features can serve to reduce or desirably channel or control planar fluid migration, and facilitate fluid movement downwardly into the product structure, to underlying absorbent layers. Further, a suitably-textured topsheet can feel more comfortable against the skin, and provide enhanced breathability, for enhanced feelings of softness, coolness and dryness.

For these reasons, assemblies or subassemblies of absorbent hygiene products that include topsheets together with conventional absorbent materials (i.e., cellulosic fiber or pulp, etc.), are often embossed to impart 3D texture to the wearer-facing surface. The 3D texture of the embossments can be configured and imparted to be quite visible to the naked eye. In some examples, an assembly including both the topsheet material and one or more underlying absorbent layers (such as the fibrous secondary topsheet and/or other underlying fibrous absorbent layer) is passed through the nip between a pair of embossing rollers, wherein at least the topsheet-side roller outer surface is formed with a desired pattern of embossing protrusions and recesses; in some examples the rollers each may have protrusions and recesses wherein the protrusions and recesses of each roller are configured to be complementary and mating with those of the other roller. When the assembly is passed through the nip, the desired pattern is impressed into the assembly including the topsheet and underlying fibrous layer(s), and is substantially retained and visible on the topsheet side of the end product.

In a more modern configuration, relatively thin, low bulk, but still effectively absorbent feminine hygiene pads having an absorbent layer formed of open-cell foam have been marketed. Examples include particular offerings of ALWAYS brand pads manufactured and sold by The Procter & Gamble Company (Cincinnati, Ohio). These products are appreciated by some market segments because they have good absorption performance, while being relatively thin (i.e., relatively low-caliper) and quite resilient and pliable, making them comfortable to wear/use, and making them relatively discreet under outer clothing. Such pads are currently offered in a configuration having a topsheet formed of a relatively thin and untextured, low-basis weight nonwoven web material.

A feature of these foam-based pads is that they do not include a fibrous layer (sometimes called a “secondary topsheet,” “acquisition layer,” or “distribution layer”) beneath the topsheet. In more traditional pulp-based pads, such a fibrous layer is often formed of a relatively higher-basis-weight section of fibrous nonwoven web material, or a batt of fibers. For foam-based pads, however, toward the objective of providing a relatively thin, pliable pad, however, such an added fibrous layer beneath the topsheet is desirably omitted.

An absorbent product having a layer of open-cell foam directly beneath a topsheet, no secondary topsheet, is typically not suitable for successful multi-layer embossing as described above. Because the foam is resilient, it may not permanently deform in the embossing process, and thus does not retain the embossing. Alternatively, if embossing pressure is too great, the foam structure may be crushed and fractured, compromising its structural integrity and potentially leading to quality perception issues. To the extent that the topsheet may retain some z-direction deformation from the embossing process, the resilient foam layer, tending to resume its pre-embossing (planar/flat) shape following its emergence from the nip between a pair of embossing rollers, will push the deformed topsheet upward and away from itself, creating gaps or spaces between the topsheet and underlying components, resulting from the variations in texture topography on the underside of the topsheet imparted by the embossing process.

Alternatively, if the topsheet material is embossed alone prior to its combination with the foam layer, similar gaps or spaces between the embossed/textured topsheet material and the foam layer may be present when the topsheet material is combined with the foam layer to form a pad.

It has been learned that, for an absorbent system of a feminine hygiene pad to function effectively, it must be able to move fluid rapidly down through the topsheet to the absorbent structure beneath. A topsheet formed of fibrous nonwoven material wicks discharged fluid along surfaces of its fiber components, and through the inter-fiber passageways among the fibrous matrix. Thus, a substantial gap or air space between a topsheet and an underlying absorbent layer will hinder movement/transfer of fluid from the topsheet to the absorbent layer.

Accordingly, for foam-based pads having a topsheet of fibrous nonwoven material and no secondary topsheet, it is desirable to have as much surface area of the underside of the topsheet and the top side of the foam absorbent layer in direct contact as possible, so that discharged fluid moving down through the topsheet and along the surfaces of the fiber constituents thereof, will reach and contact the absorbent layer quickly following discharge. Toward this objective, to date, nonwoven topsheets included with thin foam-based pads have been produced with relatively flat, unfeatured/untextured surfaces and have been of relatively thin/low-caliper themselves, so as to maximize contact surface area between topsheet and absorbent layer. In combination or alternatively, such topsheets have been formed of relatively low basis weight nonwoven web materials, with relatively low fiber count density per unit surface area, and a lack of textural features, so as to provide a topsheet that is as porous/permeable as possible while still having sufficient mechanical robustness (i.e., tensile strength) to run successfully through the pad manufacturing process (i.e., web handling) equipment.

For the reasons discussed above, topsheets included with low-caliper feminine hygiene pads, having open-cell foam absorbent layers, to date, have been relatively lacking in 3D textural features.

Accordingly, opportunity remains for development of an absorbent pad having a combination of features including those associated with current foam-based pads that are appreciated by consumer market segments, while having a relatively highly textured topsheet that provides for enhanced consumer perception, in a combination that does not compromise rapid fluid acquisition.

### BRIEF DESCRIPTION OF THE DRAWINGS

FIG. 1 is a plan view of an example of a feminine hygiene pad, topsheet side facing the viewer.

FIG. 2 is a plan view of an example of an absorbent layer.

FIG. 3A is a schematic lateral cross section of the feminine hygiene pad of FIG. 1.

FIG. 3B is an enlarged portion 3B of the drawing of FIG. 3A, enlarged to depict sublayers of an absorbent layer, which may be present in some examples.

FIGS. 4A-4C are plan views of several examples of adhesive deposit patterns within a bonding region, in which a topsheet may be bonded to an absorbent layer.

FIG. 5 is a plan view, magnified photograph of a portion of an example of a PFA nonwoven web material.

### DESCRIPTION OF EXAMPLES

### Definitions

With respect to a feminine hygiene pad that is opened and laid out flat on a horizontal planar surface, “lateral” refers to a direction perpendicular to the longitudinal direction and parallel the horizontal planar surface.

With respect to a feminine hygiene pad that is opened and laid out flat on a horizontal planar surface and having a length measured from its forward end to its rearward end, “longitudinal” refers to a direction parallel with the line along which the length is measured, and parallel to the horizontal planar surface. “Length” refers to a dimension measured in the longitudinal direction.

With respect to a feminine hygiene pad, the terms “front,” “rear,” “forward” and “rearward” relate to features or regions of the pad corresponding to the position they would occupy when the pad is ordinarily worn by a user, and the front/anterior and rear/posterior of the user's body when standing.

With respect to a feminine hygiene pad that is opened and laid out flat on a horizontal planar surface, or a nonwoven web material (for example, a topsheet of nonwoven web material) laid out flat on a horizontal planar surface, “z-direction” refers to a direction perpendicular to the horizontal planar surface, and any plane parallel to the horizontal planar surface may be referred to as an “x-y plane”. When the pad is being worn by a user (and thus has been urged into a curving configuration), “z-direction” at any particular point location on the pad refers to a direction generally normal to the wearer-facing surface of the pad at the particular point location. With respect to a nonwoven web during its manufacture, “z-direction” refers to a direction orthogonal to both the machine direction and the cross direction of manufacture, and any plane parallel to the machine direction and cross direction may be referred to as an “x-y plane”.

With respect to a feminine hygiene pad, “wearer-facing” is a relative locational term referring to a feature of a component or structure of the pad that when in use that lies closer to the wearer than another feature of the component or structure that lies along the same z-direction. For example, a topsheet has a wearer-facing surface that lies closer to the wearer than the opposite, outward-facing surface of the topsheet.

With respect to a feminine hygiene pad, “outward-facing” is a relative locational term referring to a feature of a component or structure of the pad that when in use that lies farther from the wearer than another feature of the component or structure that lies along the same z-direction. For example, a topsheet has an outward-facing surface that lies farther from the wearer than the opposite, wearer-facing surface of the topsheet.

The relative location terms “inboard” and “outboard” refer to positioning of a first feature relative a second feature with respect to the lateral or longitudinal axis of a pad or layer component thereof, when both features are on the same side of the axis. For example, a first feature is laterally “inboard” of a second feature, and the second feature is laterally “outboard” of the first feature, when the first feature is closer to the longitudinal axis of the pad than the second feature. Similarly, a first feature is longitudinally “inboard” of a second feature, and the second feature is longitudinally “outboard” of the first feature, when the first feature is closer to the lateral axis of the pad than the second feature.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

### DESCRIPTION

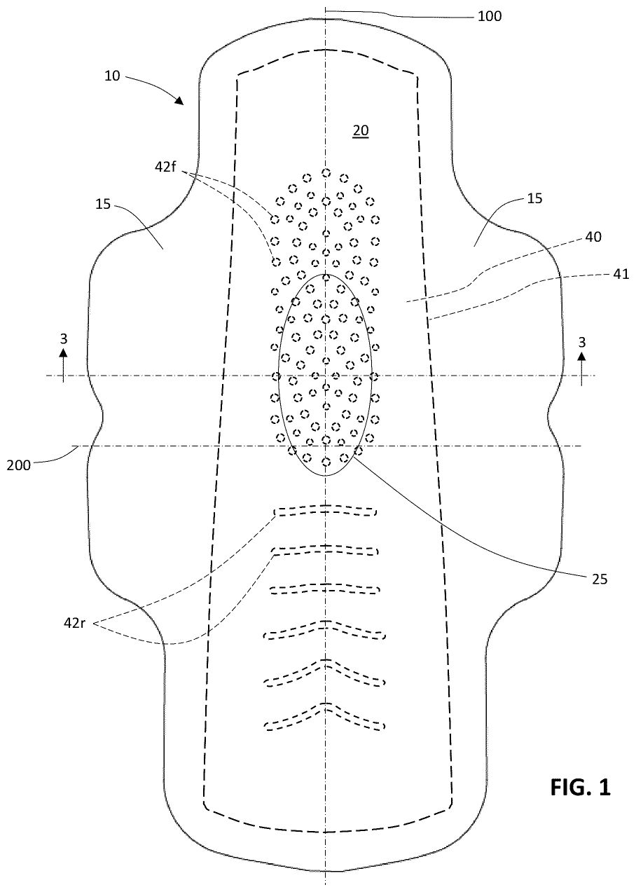

Referring to FIGS. 1, 2 and 3A, a feminine hygiene pad 10 may include a liquid permeable topsheet 20, a liquid impermeable backsheet 30 and an absorbent layer 40 disposed between the topsheet and the backsheet. The absorbent layer has an outer perimeter 41. In regions outside the outer perimeter 41, the topsheet and the backsheet may be bonded together in laminated fashion by any suitable mechanism including but not limited to adhesive bonding, thermal bonding, pressure bonding, etc., thereby enveloping, retaining and holding the absorbent layer 40 in place between the topsheet 20 and the backsheet 30. Absorbent layer 40 may be cut or otherwise imparted with a shape that is asymmetric about the lateral axis, as suggested in the figures, for purposes of enabling the cutting away of consecutive absorbent layers 40 from stock material along nested profiles that provide for efficient absorbent layer stock material utilization/minimization of cutoff scrap. It may be preferred that the laterally wider portion of the shape is in the rear of the pad, for purposes of providing more surface area to intercept discharged fluid that moves along a wearer's skin through the gluteal crevice. Pad 10 may include opposing wing portions 15 extending laterally outside of perimeter 41 by a comparatively greater width dimension than the main portion of the pad. The outer surface of the backsheet forming the undersides of the main portion and the wing portions may have deposits of adhesive 35 thereon. Adhesive deposits 35 may be provided to enable the user to adhere the pad to the inside of her underpants in the crotch region thereof, and wrap the wing portions through and around the inside edges of the leg openings of the underpants and adhere them to the outside/underside of the underpants in the crotch region, providing supplemental position holding support and helping guard the leg edges of the underpants against soiling. When pad 10 is packaged, adhesive deposits 35 may be covered by one or more sheets of release film or paper (not shown) that covers/shields the adhesive deposits 35 from contact with other surfaces until the user is ready to remove the release film or paper and place the pad inside her underpants for use.

### Topsheet

Topsheet 20 may be formed of a suitable nonwoven web material that is compliant, soft feeling, and non-irritating to wearers' skin. Referring back to the figures, the topsheet 20 is positioned adjacent a wearer-facing surface of the absorbent layer 40 and may be joined thereto and to the backsheet 30 by any suitable attachment or bonding method. The topsheet 20 and the backsheet 30 may be joined directly to each other in the peripheral regions outside the perimeter 41 of the absorbent layer 40 and may be indirectly joined by directly joining them respectively to wearer-facing and outward-facing surfaces of the absorbent layer or additional optional layers included with the pad.

A suitable topsheet material will include a liquid pervious material that is comfortable when in contact with the wearer's skin and under suitable circumstances will permit discharged menstrual fluid to rapidly move through it. A suitable topsheet may be made of various materials such as nonwoven web materials.

As contemplated herein, component nonwoven web material from which topsheet 20 may be cut may be a nonwoven web material that includes or consists predominately (by weight) of fibers spun from polymeric resin such as polyolefins and/or polyesters, including but not limited polypropylene, polyethylene, polyethylene terephthalate (PET) and variants, blends, and bicomponent or multicomponent arrangements thereof. Topsheet component nonwoven web material also may include in partial or predominant weight fraction natural fibers such as but not limited to cotton, hemp, kapok, bamboo, etc.

The nonwoven web may be formed via any suitable process by which spun fibers of indefinite and/or staple lengths may be distributed and accumulated in a controlled fashion onto a moving forming belt to form a batt having a desired distribution of the fibers, to a desired basis weight. Suitable processes may include spunbonding and meltblowing (for fibers of indefinite, relatively greater length), and carding or airlaying (for fibers of staple, relatively shorter length) or co-forming (for a mix of fibers of indefinite lengths and fibers of staple lengths). After accumulation, the batt may be processed to consolidate and bond the fibers into a cohesive web by any suitable method, including calendering, calender thermal bonding, calender compression bonding, through-air bonding, etc. The batt or consolidated web may also be subjected to processes such as hydroenhancing or hydroentangling, to impart web cohesiveness via inter-fiber entanglement, increase z-direction orientation of fibers, and/or increase loft.

Absent enhancements to the materials and/or processes involved, generally, monocomponent fibers spun from polymer resin tend to have relatively simple surface geometry, typically an approximately round or oval-shaped cross section, and a substantially non-curled or non-crimped configuration along the lengths thereof. As a consequence, when the spun fibers are deposited and accumulated on a forming belt, calendered and bonded (e.g. in a spunbonding process), the resulting nonwoven web product will have relatively low loft and a relatively macroscopically flat and untextured appearance, as compared with a web of a comparable basis weight formed of more complexly-shaped, e.g., curled or crimped, fibers. Some consumers may perceive a relatively lower loft nonwoven web to have a relatively less pleasing feel and appearance, i.e., it may perceived to be, relatively, not as soft or luxurious, as a higher-loft one.

To add loft to the web without increasing basis weight (and material usage), and to increase opacity of the web, the fibers used to make the web may be spun in multicomponent, e.g. bicomponent, fiber configurations. Resin-processing equipment and beams of spinnerets may be configured, and polymer resins may be selected, to spin bicomponent fibers that crimp or curl as they leave the spinnerets as molten polymer streams, and subsequently cool and solidify into fibers. Known processes and polymer resin selections may be used to produce curly spun bicomponent fibers wherein the fibers have side-by-side, eccentric core-sheath, or other non-coaxial polymer component cross-sectional configurations. In such non-coaxial configurations, one of the polymer components may be selected and/or formulated to have a differing melting temperature and/or cooling contraction rate than the other polymer component. Upon cooling, the differing properties of the polymer components and non-coaxial cross-sectional arrangement of component sections of the molten fiber streams impart curl to the fibers as they cool, contract at differing rates and solidify. The respective polymer resin components may be differing polymers, differing forms or variants of the same polymers, or differing blends thereof. More detailed disclosure of spinning curled or crimped bicomponent fibers and forming a nonwoven web thereof may be found in, for example, U.S. Pat. No. 8,501,646; EP 1 988 793; and US 2007/0275622. In some examples bicomponent fibers may have respective predominately polypropylene-based resin components formulated to impart differing melting temperatures to the respective components. In some examples bicomponent fibers may have respective components in which one component is predominately polypropylene-based, and the other component is predominately polyethylene-based. In some more particular examples the bicomponent fibers may be spun with an eccentric core-sheath component configuration wherein a predominately polypropylene-based component is the core component and a predominately polyethylene-based component is the sheath component; wherein the polypropylene-based component may be desired for its greater tensile strength, and the polyethylene-cased component may be desired for its smoother, more lubricious surface feel, that helps impart a silky feel to the fiber and to the nonwoven web material. It will be appreciated that other combinations of polyolefins and/or other spinnable thermoplastic resins may be selected for their differing cooling contraction rates and other differing qualities that affect the qualities (including curl or crimp) and properties of the spun fibers in differing ways.

Another potential advantage of inclusion of bicomponent fibers is that, with appropriate selection of the component resins for the fibers, the fibers in the web may be bonded to each other at random locations via application of heat or heated air-through bonding, wherein the fibers are heated to an extent sufficient to cause them to partially melt and fuse together at locations where they contact each other. The fiber component resins may be selected such that a first component resin has a lower melting temperature than the second component resin. The web may be heated to an extent sufficient to cause the first component resin but not the second component resin to partially melt, such that the first components of contacting fibers will fuse, without causing the second components to lose their shape. In this manner, the web may imparted with added resiliency and mechanical (tensile) strength, without a loss of z-direction caliper/loft that would be otherwise caused by z-direction compression as occurs in other bonding processes such as, for example, calender bonding. In some examples, bicomponent fibers having a sheath-core configuration may be provided with a sheath component having a relatively lower melting temperature and a core component having a relatively higher melting temperature. Using a heat treatment as described above, the web may be bonded such that the sheaths of the fibers fuse, without melting of the cores. In some examples, the sheath components may be formed of or include a polyethylene having a lower melting temperature, and the core components may be formed of or include a polypropylene or a polyethylene terephthalate (PET) having a higher melting temperature.

The topsheet may further incorporate or include any features of topsheets described in U.S. patent application Ser. Nos. 16/789,516) and/or 16/789,522.

Many commercially practical thermoplastic resins that may be desired to process and spin into bicomponent fibers are normally hydrophobic. Such resins may include polyolefins such as polypropylene and polyethylene. A nonwoven web material formed of such fibers will also be hydrophobic, and as such, will not readily accept or wick aqueous fluid such as menstrual fluid. When such resins are used, therefore, additional measures can be included to render the fibers and/or the nonwoven web, or portions thereof, hydrophilic. In some examples, a suitable surfactant may be applied to the nonwoven web following its formation. In more particular examples, a suitable surfactant finish used may be SILASTOL PHP 26, a product of Schill+Seilacher GmbH, Böblingen, Germany. Other suitable surfactants may include STANTEX spin finishes (Pulcra Chemicals, Geretsried, Germany), for example, STANTEX 6887; other SILASTOL spin finishes, for example PHP26, PHP28, PHP90, PHP207, PST-N, etc. In addition or alternatively, resin-incorporated surfactants (i.e., melt additives) may be added to polymer resin prior to fiber spinning, which can impart and/or increase hydrophilicity. Suitable melt additives may include PPM15560, PM19668 and PPM112172 (Techmer PM, Clinton, Tennessee), or VW351 or S-1416 (Polyvel Inc., Hammonton, New Jersey). Staple fibers in carded webs may have spin finishes provided as supplied to impart the desired surface energy as well as other characteristics.

In some examples, it may be desired to selectively render or treat fibers of portions of the topsheet nonwoven web material with agents or additives to impart a combination of enhanced hydrophilicity and enhanced hydrophobicity, according to particular spatial, i.e., z-direction and/or x-y planar locations or regions of the topsheet. For example, it may be desired that fibers proximate the wearer-facing top side of the web be predominately hydrophobic, and that fibers proximate the underside of the topsheet be predominately hydrophilic. In such examples, this may be desired to cause underlying fibers to attract and draw fluid from overlying fibers, and wick the fluid down to the underlying absorbent layer, while causing overlying fibers to be more resistive to rewetting by movement of fluid back up to the wearer-facing surface. In such examples, the fibers of the web may be inherently hydrophobic and/or imparted with enhanced hydrophobicity by inclusion of hydrophobicity-increasing melt additives in the component polymer resin, prior to spinning. Suitable melt additives may include, for example erucamide or glycerol tristearate. Following manufacture, the underside of the topsheet material may be selectively treated, across its entire area, or a sub-portion thereof, with a hydrophilicity-enhancing agent. Non-limiting examples are described in US App. Pub. No. 2019/0388578.

The finish may be applied to the web using any suitable method, for example, via kiss roll coater. The finish may be applied in a quantity suitable to impart the nonwoven web with a desired level of hydrophilicity and thereby help impart it with a desired wicking capability. In particular examples, a finish coating of SILASTOL PHP 26 may be applied in a quantity sufficient to constitute, after drying, surfactant weight quantity that is 0.30 percent to 0.60 percent, more preferably 0.40 percent to 0.50 percent of the basis weight of the nonwoven web material.

Wicking performance also may vary according to, and may be manipulated by, the manner in which the web is further processed. Factors such as level of consolidation (i.e., densification) of the fiber mass in the end structure and orientations of the individual fibers within the end structure can affect absorbency and wicking performance.

Thus, for purposes contemplated herein, in combination with being imparted with a suitable basis weight, density and/or caliper as discussed above, it may be desired that nonwoven web material formed in part or in substantial entirety of fibers spun from thermoplastic polymer resin and used to make the topsheet, be formed via a nonwoven web manufacturing process in which substantial portions of the fibers are imparted with directional orientation that includes some z-direction orientation, rather than orientations predominately biased along the machine direction or x-y plane of formation of the web structure. Following any suitable processes in which fibers are distributed and laid down in a batt on a horizontal forming belt (e.g., via a spunbond process), additional process steps that forcibly reorient some of the fibers or portions thereof in the z-direction may be employed. Suitable process steps may include needle punching and hydroentangling or hydroenhancing. Hydroentangling or hydroenhancing, in which an array of fine, high-velocity water jets is directed at the batt as it is conveyed past them, may be desired for its effectiveness in reorienting lengths of fibers while breaking fewer fibers and creating less broken fiber lint and surface fuzz (free fiber ends extending from the surface of the web). The batt is conveyed past the water jets on a belt or drum having a surface constituted by a permeable fine mesh or screen having a pattern of orifices or pores, through which the jetted water may be drawn. The belt or drum surface may also include a pattern or other configuration of protrusions and/or recesses arranged to cause the web to be imparted with textural features in the water-jetting step. A vacuum water removal system may be operably disposed on the back side or underside of the belt or drum surface, to draw the jetted water therethrough, and can tends to create, add, open and/or clear small z-direction passageways within the fiber matrix of the web, approximately in the pattern of the orifices or pores. Without intending to be bound by theory, it is believed that the portions of the fibers oriented in the z-direction and the z-direction passageways increase the ability and tendency of the web to wick aqueous fluid in the z-direction. In a topsheet, this would mean that the material can more readily wick aqueous fluid from the wearer-facing surface of the topsheet to the outward-facing surface of the topsheet, i.e., down to the absorbent layer below, and may thereby wick fluid less along x-y planar directions (causing a stain from discharged fluid to spread laterally and/or longitudinally).

### Nonwoven Topsheet Texture Characteristics

As noted in the Background, it is believed that consumers/users of absorbent articles of the types contemplated herein (including feminine hygiene pads) appreciate visible 3D texture in the visible wearer-facing surface of the product. However, it is believed that, to date, a thin and pliable feminine hygiene pad that has a nonwoven web topsheet directly overlying a layer of absorbent foam to serve functions of receiving and absorbing discharged menstrual fluid, has not been offered with a topsheet having appreciable, visible 3D texture. It is believed that, prior to the research and experimentation described herein, it was generally believed in the art that a textured nonwoven topsheet (myriad examples have been manufactured and are available for purchase) would be unsuitable for combination with directly subjacent foam absorbent layer, because it would compromise fluid acquisition performance.

It has been learned, however, that a nonwoven web material exhibiting a desired 3D patterned texture pattern on one side thereof, while being relatively less-textured or even substantially less-textured, non-textured or flat on the other side, may be manufactured. Through prototyping and experimentation it has been learned that such a material can be successfully combined with an open-cell foam absorbent layer, wherein the textured side is disposed to the wearer-facing side of the article, and the relatively less-textured side is disposed to face the foam absorbent layer. In connection with this effort, techniques have been discovered that enable the characterization and identification of suitable nonwoven web material, and distinguishing a textured nonwoven web material that is suitable, from others that are unsuitable. By “suitable” in this context, it is meant that the nonwoven web material has 3D texture or topographic features on one side that are readily visible to the naked eye, while having a relatively less-textured side that does not unacceptably compromise the ability of the combination of topsheet and foam to receive and absorb discharged menstrual fluid at an acceptable rate.

In a series of patent applications that include those having publication numbers: US2017/0027774; US2017/0029993; US2017/0191198; WO2017/105997; US2017/0029994; US2018/0168893; WO2018/112144; WO2018/112146; US2018/0216269; US2018/0216271; US2018/0216270; US2018/0214318; US2020/0268572; US2019/0003079; US2019/0003080; US2019/0374405; US2019/0374407; US2019/0374388; US2019/0380887; US2019/0298587; US2019/0298586; US2020/0299880; US2020/0299881; US2020/0345563; US2020/0347533; US2020/0397629; US2020/0397630; US2020/0100956; US2021/0169710; US2021/0369511; US2021/0369512; and US2022/0192897, and PCT/CN2023/073146, methods for manufacturing nonwoven web materials with formed, ordered patterns of alternating built-up regions and attenuated regions are disclosed. These methods do not include or require embossing of the web material. As explained and depicted in more detail in, for example, US2019/0380887, U.S. Pat. Nos. 10,765,565 and 11,547,613, the built-up regions are regions of the nonwoven material in which constituent spun fiber count and numerical density per unit x-y planar surface area are relatively greater; and the attenuated regions are regions in which constituent spun fiber count and numerical density per unit x-y planar surface area are relatively lesser. The regions of built-up fiber count and numerical density also have visibly relatively greater z-direction caliper, while the attenuated regions have visibly relatively lesser z-direction caliper. The forming belt or drum used in the method may be adapted so as to impart a desired pattern to the built-up regions and attenuated regions, and thus, desired visible textural features.

Generally, this nonwoven structure may be manufactured by entraining continuous spun polymer filaments (i.e., continuous fibers—herein the terms “filaments” and “fibers” are used interchangeably) into a continuous air stream. The air stream and thus the entrained filaments are directed to a working location on a nonwoven web manufacturing line, at which a continuously cycling forming belt or rotating forming drum are located. The receiving surface of the cycling forming belt or rotating forming drum cycles and travels through the working location, along a machine direction. The receiving surface of the forming belt or drum has an ordered pattern of airflow-permeable regions and airflow-blocked regions formed thereon. The air-flow permeable regions are formed by a screen-type structure with apertures or pores that are numerous enough to allow air to pass through the receiving surface relatively freely, and small enough to substantially prevent entrained filaments from being carried through with the air. A vacuum system is disposed opposite the receiving surface and draws the air in the air stream through the airflow-permeable regions. As a result, as the entrained filaments are carried toward the receiving surface, and they are drawn to, strike, and are accumulated on, the receiving surface, they accumulate more heavily over the air-permeable regions, and less heavily over the airflow-blocked regions on the receiving surface. The resulting batt having built-up regions and attenuated regions of accumulated filaments is then separated from the receiving surface for further processing downstream. The areas of heavier accumulation (built-up regions) and areas of lighter accumulation (attenuated regions) correspond to the ordered pattern of airflow-permeable regions and airflow-blocked regions formed on the receiving surface of the belt or drum. It has been learned that, when the receiving surface of the forming belt or drum is suitably configured such that the airflow-permeable regions are located within pockets or surface depressions and the airflow-blocked regions include raised areas or protrusions, the resulting formed nonwoven batt will be “sided,” having a substantial macroscopic 3D texture on the side that was furthest from the air/entrained filament source (consisting more of filaments that were accumulated earlier in time, more heavily in the airflow permeable regions of the receiving surface), and substantially reduced macroscopic 3D texture (greater flatness) on the side that was closest to the air/entrained filament source (consisting more of fibers that were accumulated later in time, to overlie the earlier-accumulated filaments). Calendering of the batt and calender bonding, if desired, in downstream processes, can help to further flatten the less-textured/flatter side. For purposes herein, a nonwoven material manufactured in this manner will be referred to as a “Patterned Fiber Accumulation” (PFA) material.

It has been learned through experimentation that a topsheet made from a nonwoven web material manufactured in this manner may be combined with an absorbent foam layer, to form an absorbent article such as a feminine hygiene pad, with a wearer-facing topsheet surface having desirable macroscopic 3D texture or topographical features, wherein the ability of the pad to receive and absorb menstrual fluid at a sufficient rate is still achieved. Since fluid moves through a topsheet nonwoven material by flowing along surfaces of constituent fibers, it is desirable to ensure that as many fiber surfaces as possible are in contact amongst themselves and directly or indirectly with fiber surfaces contacting with the upper surface of the subjacent foam layer, so that the fluid will rapidly wick along fiber surfaces down through the topsheet, and then contact the foam layer and be drawn into it. A relatively flatter lower topsheet surface facing a subjacent foam absorbent layer will provide for relatively greater contact area between the fibers defining the lower topsheet surface, and the foam layer.

In connection with this effort and discovery, methods for characterizing desired 3D texture features of a nonwoven web material, which forms a topsheet of an absorbent article, have been identified. Given the myriad 3D texture designs that might be imagined and implemented through configuration of nonwoven web forming equipment, the methods enable the identification of a material that will be successful in presenting a visible, macroscopic 3D texture to the user, while exhibiting parity with or improvement on the fluid handling characteristics (fluid receiving and absorption performance, and low rewetting) of currently-marketed, foam-based pads that lack substantial 3D topsheet texture. These methods are set forth below, and are used to measure “Sdc” and “Sdr %”.

Generally, the measured value of Sdc reflects an average magnitude of a difference in z-direction height between highest points and lowest points of the topography of one side of a nonwoven web material. Thus, a measured Sdc of a higher magnitude reflects a more dramatically textured topography in terms of height differences between highest “peaks” and lowest “valleys” of the topography.

Generally, the measured value of Sdr % reflects the difference between macroscopic x-y planar surface area as viewed along a z-direction, and surface area of all of the topographic features, along all surface contours they define. It is expressed as a percent ratio of surface area along surface contours, to macroscopic x-y planar surface area. To illustrate the concept, a mountain on Earth has a horizontal/projected two-dimensional x-y planar surface area as viewed directly along a vertical (z) direction from space, but also contour surface area in three dimensions (3D), theoretically measurable by physically walking along, measuring and tallying the area along all contours that its land surface defines. The steeper and/or more complex the surface topography of the mountain, the greater is its ratio of its contour surface area to its horizontal/projected x-y planar surface area; conversely, the flatter and/or more simple the surface topography of the mountain, the lesser is its ratio of its contour surface area to its horizontal/projected x-y planar surface. Thus, for a topsheet, the ratio quantifies the extent to which topsheet 3D topography/contour surface area differs from that of a horizontal/projected plane of the same x-y dimensions. For a topsheet nonwoven, the measured and calculated ratio that follows this analysis is Sdr %.

Because the “surface”, or side, of a nonwoven is actually made up, at a microscopic level, of the many portions of surfaces of the fibers that are outermost in the z-direction proximate that side, it will be appreciated that these measurement methods and the values measured will depend to some extent upon the resolution size selected for the imaging equipment used in the measurement method. If the selected resolution is too high, the measurements will reflect differences in positions among individual fibers throughout the entire z-direction caliper of the nonwoven, rather than more macroscopic surface contours/texture (or flatness) of one side of the material. Conversely, if the selected resolution is too low, the measurements will not make sufficient distinction between a highly textured surface and a relatively flat one, to enable a determination whether a particular nonwoven material is suitable for purposes contemplated herein. Accordingly, for purposes herein, a resolution size has been selected for use in the measurement methods, which is deemed appropriate for characterizing and measuring topsheet topography to generate data deemed suitable to reflect visibility of enhanced topsheet topography to the naked eye, and to reflect flatness of topsheet topography for purposes effectuating rapid transfer of fluid from the topsheet to an underlying absorbent foam layer.

From prototyping and experimentation with various topsheet materials including PFA material produced by the process described above as well as examples of spunbond and carded/spunlaced/hydrojetted materials imparted with surface texture via hydrojetting over drums with patterned forming surfaces, it has been determined that a combination of topsheet nonwoven with an absorbent foam layer directly therebeneath (i.e., subjacent), will be attractive to consumers who prefer topsheet texture, while performing at parity or better as compared with current market products with respect to fluid acquisition and absorption rates, when the foam layer wearing-facing (upper) surface, the topsheet outward-facing (lower) surface, and the topsheet wearer-facing (upper) surface are manufactured such that their measured values for Sdc and Sdr % conform to the following limits:

TABLE 1

| | Sdc (mm) | Sdr % |
|---|---|---|
| Topsheet upper | >0.360 | >1.0 |
| surface | more preferably >0.420 | more preferably >1.1 |
| Topsheet lower | <0.350 | <0.80 |
| surface | more preferably <0.300 | more preferably <0.750 |
| Foam layer upper | <0.200 | <0.100 |
| surface | more preferably <0.150 | more preferably <0.050 |

A foam layer exhibiting values conforming to the limits for Sdc and Sdr % shown in Table 1, above, may be manufactured as described below.
Alternatively, or in combination, the values reflecting the magnitude of texture/contouring of the topsheet lower surface may be kept within a maximum difference exceeding that of the foam layer upper surface, as follows:

TABLE 2

| | ΔSdc (mm) | ΔSdc % |
|---|---|---|
| Topsheet lower | <0.250 | <250 |
| surface | more preferably <0.200 | more preferably <200 |

In Table 2, “ΔSdc” is the difference between the measured value of Sdc for the lower surface of the topsheet material, and the measured value of Sdc for the upper surface of the absorbent foam layer. “ΔSdc %” is the ratio of ΔSdc to the measured value of Sdc for the upper surface of the absorbent foam layer, times 100%.

TABLE 3

| | ΔSdr % | ΔSdr % % |
|---|---|---|
| Topsheet lower | <1.00 | <5,000 |
| surface | more preferably <0.800 | more preferably <3,500 |

In Table 3, “ΔSdr %” is the difference between the measured value of Sdr % for the lower surface of the topsheet material, and the measured value of Sdr % for the upper surface of the absorbent foam layer. “ΔSdr % %” is the ratio of ΔSdr % to the measured value of Sdr % for the upper surface of the absorbent foam layer, times 100%.

Through research and prototyping, successful and unsuccessful examples of particular combinations of topsheet materials and absorbent foam have been identified. In each combination shown in Table 4 below, the absorbent foam layer was the HIPE foam layer used in current ALWAYS INFINITY brand feminine hygiene pads, manufactured by The Procter & Gamble Company, Cincinnati, Ohio. The control product was an example of current market ALWAYS INFINITY pads with non-textured nonwoven topsheets.

The upper (topsheet-facing) surface of the foam layer exhibited values for Sdc and Sdr % of 0.104 and 0.022, respectively. For each combination, the topsheet was bonded with adhesive to the subjacent foam layer via a pattern of applied adhesive substantially the same as that appearing in the control product.

The topsheets tested exhibited the following values for Sdc and Sdr % (Table 4). Several combinations were deemed successful at presenting a visually appealing topsheet surface texture, while maintaining parity or better fluid acquisition speed and low rewetting, as compared to the control product:

TABLE 4

| Material | Topsheet Upper Side | | Topsheet Lower Side | | |
|---|---|---|---|---|---|
| Example | Sdc (mm) | Sdr % | Sdc (mm) | Sdr % | Result |
| 1 | 0.437 | 1.190 | 0.297 | 0.733 | Success |
| 2 | 0.368 | 1.337 | 0.284 | 0.726 | Success |
| CONTROL | 0.146 | 0.056 | 0.117 | 0.060 | |
| 3 | 0.539 | 2.214 | 0.271 | 0.584 | Success |
| 4 | 0.401 | 1.196 | 0.385 | 1.182 | Failure |
| 5 | 0.721 | 3.347 | 0.641 | 2.926 | Failure |

Material Example 1 was a 25 gsm calendar-bonded spun-bond nonwoven web formed of bicomponent fibers in a side-by-side configuration, obtained from Berry Global Inc., Evansville, Indiana. The components were polypropylene and co-polypropylene, in a 1:1 weight balance. The average fiber diameter was about 20 μm. The fibers were curled or crimped as a consequence of their respective components and configuration. The upper surface bore texture imparted by a hydro-jetting process specified by the manufacturer, such that the lower surface reflected comparatively less texture.

Material Example 2 was a 27 gsm web having two layers of carded staple fibers, obtained from Xiamen Yanjan New Material Co. Ltd., Xiamen, China. The upper layer was 11 gsm and was formed of 1.5 denier bicomponent fibers of a sheath-core configuration, wherein the sheath component was polyethylene and the core component was PET, in a 1:1 weight balance. The top layer fibers were treated with a spin finish that imparted hydrophobicity. The lower layer was 16 gsm and formed of 3.0 denier bicomponent fibers of a sheath-core configuration, wherein the sheath component was polyethylene and the core component was polypropylene, in a 1:1 weight balance. The fibers of the lower layer were surface-treated prior to carding, to render them hydrophilic. The web was through-air bonded to consolidate it and impart cohesiveness and mechanical integrity. Texture was imparted via passage of the web through the nip between a pair of mating, heated rollers having operative surfaces with respective complementary patterns of pins and mating holes.

The CONTROL material was a 25 gsm spunbond nonwoven web obtained from Fibertex Nonwovens LLC (Greenville, North Carolina), having one layer of polypropylene monofilaments and two layers of crimped side-by-side bicomponent polypropylene filaments. The web additionally included 0.45 percent by weight of a topical surfactant (SILASTOL PHP26 from Schill & Seilacher GmbH), applied to the underside (outward-facing side) of the material. (The topsheet of current ALWAYS INFINITY brand feminine hygiene pads (The Procter & Gamble Company, Cincinnati, Ohio) is made of this material.)

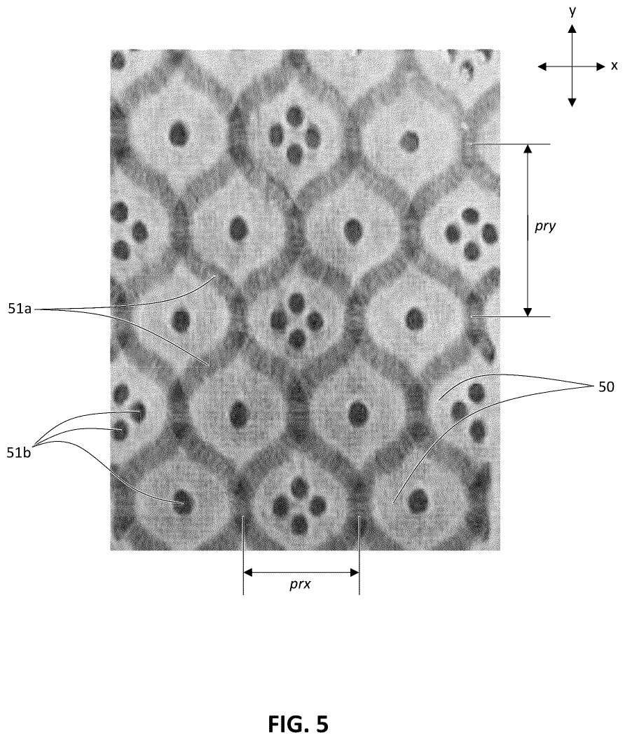

Material Example 3 was a PFA material manufactured as described herein. It was a 35 gsm air-through bonded spunbond nonwoven web. The web was formed of bicomponent fibers having a sheath/core configuration with 1:1 polyethylene/polypropylene weight balance, wherein polyethylene formed the fiber sheaths and polypropylene formed the fiber cores. The fibers were spun to have an average diameter of 16 μm. The web additionally included 3.1% by weight of a topically-applied surfactant (STANTEX S6887 from Pulchra Chemicals GmbH), applied to the underside (outward-facing side) of the material. The material was manufactured to have a pattern of built-up regions 50 and attenuated regions 51a, 51b shown in FIG. 5. The pattern of attenuated regions 51a had a repeat distance prx along the x-direction of approximately 18 mm and a repeat distance along the y-direction pry of approximately 30 mm.

Material Example 4 was a two-layered, 35 gsm material of carded staple fibers obtained from Xiamen Yanjan New Material Co. Ltd., Xiamen, China. The upper layer was 10 gsm and was formed of 1.5 denier bicomponent fibers of a sheath/core configuration in which the sheath component was polyethylene and the core component was PET, in a 1:1 weight balance. The fibers of the upper layer were left in their naturally hydrophobic state. The lower layer was 25 gsm and was formed of 2.5 denier bicomponent fibers of an eccentric sheath/core configuration in which the sheath component was polyethylene and the core component was PET, in a 1:1 weight balance. The fibers of the lower layer were surface-treated prior to carding, to render them hydrophilic. The web was through-air bonded to consolidate it and impart cohesiveness and mechanical integrity. Texture was imparted via passage of the web through the nip between a pair of mating, heated rollers having operative surfaces with respective complementary patterns of pins and mating holes.

Material Example 5 was a two-layered, 30 gsm material of carded staple fibers obtained from Xiamen Yanjan New Material Co. Ltd., Xiamen, China. The upper layer was 10 gsm and was formed of 1.5 denier bicomponent fibers of a sheath/core configuration in which the sheath component was polyethylene and the core component was PET, in a 1:1 weight balance. The fibers of the upper layer were left in their naturally hydrophobic state. The lower layer was 20 gsm and was formed of 2.5 denier bicomponent fibers of a sheath/core configuration in which the sheath component was polyethylene and the core component was PET, in a 1:1 weight balance. The fibers of the lower layer were surface-treated prior to carding, to render them hydrophilic. The web was through-air bonded to consolidate it and impart cohesiveness and mechanical integrity. Texture was imparted via passage of the web through the nip between a pair of mating, heated rollers having operative surfaces with respective complementary patterns of pins and mating holes.

It is believed that the successful fluid acquisition performance of topsheets made of Material Examples 1, 2 and 3, which exhibited substantial visible texture/top surface topography, is attributable to the relatively lower values for Sdc and Sdr % exhibited by these materials, reflecting relatively flatter lower surfaces facing the absorbent layer, and therefore, relatively greater surface contact area therebetween. Conversely, it is believed that the failure of fluid handling performance (failure to maintain parity with the control material in resistance to rewetting) of Material Examples 4 and 5 is attributable to the relatively higher values for Sdc and Sdr % exhibited by these materials, reflecting relatively more textured lower surfaces facing the absorbent layer, and therefore, relatively lesser surface contact area therebetween.

### Absorbent Layer

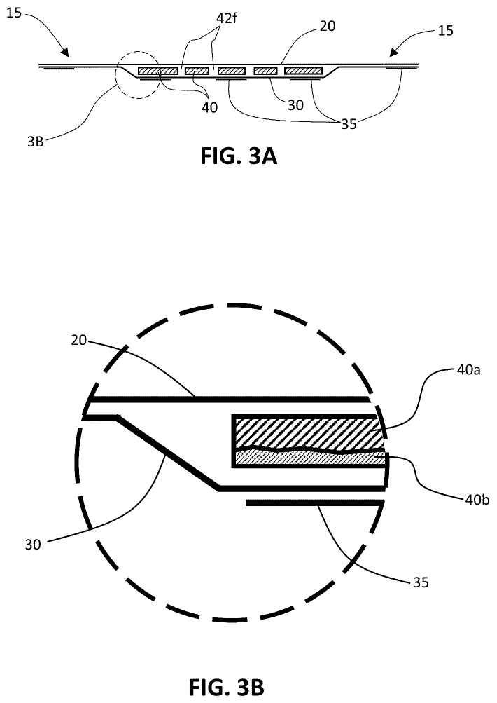

In some examples the absorbent layer 40 may be formed of or include a layer of absorbent open-celled foam material. In some examples, the foam material may include at least first and second sublayers 40a, 40b (FIG. 3B) of absorbent open-celled foam material, the sublayers being in direct face-to-face contact with each other. In such examples, the wearer-facing sublayer may be a relatively larger-celled foam material, and the outward-facing sublayer may be a relatively smaller-celled foam material, for purposes explained in more detail below.

The open-celled foam material may be a foam material that is manufactured via polymerization of the continuous oil phase of a water-in-oil high internal phase emulsion (“HIPE”).

A water-in-oil HIPE has two phases. One phase is a continuous oil phase comprising monomers to be polymerized, and an emulsifier to help stabilize the HIPE. The oil phase may also include one or more photoinitiators. The monomer component may be included in an amount of from about 80% to about 99%, and in certain examples from about 85% to about 95% by weight of the oil phase. The emulsifier component, which is soluble in the oil phase and suitable for forming a stable water-in-oil emulsion may be included in the oil phase in an amount of from about 1% to about 20% by weight of the oil phase. The emulsion may be formed at an emulsification temperature of from about 20° C. to about 130° C. and in certain examples from about 50° C. to about 100° C.

In general, the monomers may be included in an amount of about 20% to about 97% by weight of the oil phase and may include at least one substantially water-insoluble monofunctional alkyl acrylate or alkyl methacrylate. For example, monomers of this type may include C4-C18 alkyl acrylates and C2-C18 methacrylates, such as ethylhexyl acrylate, butyl acrylate, hexyl acrylate, octyl acrylate, nonyl acrylate, decyl acrylate, isodecyl acrylate, tetradecyl acrylate, benzyl acrylate, nonyl phenyl acrylate, hexyl methacrylate, 2-ethylhexyl methacrylate, octyl methacrylate, nonyl methacrylate, decyl methacrylate, isodecyl methacrylate, dodecyl methacrylate, tetradecyl methacrylate, and octadecyl methacrylate.

The oil phase may also include from about 2% to about 40%, and in certain examples from about 10% to about 30%, by weight of the oil phase, a substantially water-insoluble, polyfunctional crosslinking alkyl acrylate or methacrylate. This crosslinking comonomer, or crosslinker, is added to confer strength and resilience to the resulting HIPE foam. Examples of crosslinking monomers of this type comprise monomers containing two or more activated acrylate, methacrylate groups, or combinations thereof. Nonlimiting examples of this group include 1,6-hexanedioldiacrylate, 1,4-butanedioldimethacrylate, trimethylolpropane triacrylate, trimethylolpropane trimethacrylate, 1,1 2-dodecyldimethacrylate, 1,14-tetradecanedioldimethacrylate, ethylene glycol dimethacrylate, neopentyl glycol diacrylate (2,2-dimethylpropanediol diacrylate), hexanediol acrylate methacrylate, glucose pentaacrylate, sorbitan pentaacrylate, and the like. Other examples of crosslinkers contain a mixture of acrylate and methacrylate moieties, such as ethylene glycol acrylate-methacrylate and neopentyl glycol acrylate-methacrylate. The ratio of methacrylate:acrylate group in the mixed crosslinker may be varied from 50:50 to any other ratio as needed.

Any third substantially water-insoluble comonomer may be added to the oil phase in weight percentages of from about 0% to about 15% by weight of the oil phase, in certain examples from about 2% to about 8%, to modify properties of the HIPE foams. In certain cases, “toughening” monomers may be desired to impart toughness to the resulting HIPE foam. These include monomers such as styrene, vinyl chloride, vinylidene chloride, isoprene, and chloroprene. Without being bound by theory, it is believed that such monomers aid in stabilizing the HIPE during polymerization (also known as “curing”) to provide a more homogeneous and better-formed HIPE foam which results in greater toughness, tensile strength, abrasion resistance, and the like. Monomers may also be added to confer flame retardancy, as disclosed, for example, in U.S. Pat. No. 6,160,028. Monomers may be added to impart color (for example vinyl ferrocene); to impart fluorescent properties; to impart radiation resistance; to impart opacity to radiation (for example lead tetraacrylate); to disperse charge; to reflect incident infrared light; to absorb radio waves; to make surfaces of the HIPE foam struts or cell walls wettable; or for any other desired property in a HIPE foam. In some cases, these additional monomers may slow the overall process of conversion of HIPE to HIPE foam, the tradeoff being necessary if the desired property is to be conferred. Thus, such monomers can also be used to slow down the polymerization rate of a HIPE. Examples of monomers of this type comprise styrene and vinyl chloride.

The oil phase may further include an emulsifier to stabilize the HIPE. Emulsifiers used in a HIPE can include: (a) sorbitan monoesters of branched C16-C24 fatty acids; linear unsaturated C16-C22 fatty acids; and linear saturated C12-C14 fatty acids, such as sorbitan monooleate, sorbitan monomyristate, and sorbitan monoesters, sorbitan monolaurate diglycerol monooleate (DGMO), polyglycerol monoisostearate (PGMIS), and polyglycerol monomyristate (PGMM); (b) polyglycerol monoesters of -branched C16-C24 fatty acids, linear unsaturated C16-C22 fatty acids, or linear saturated C12-C14 fatty acids, such as diglycerol monooleate (for example diglycerol monoesters of C18:1 fatty acids), diglycerol monomyristate, diglycerol monoisostearate, and diglycerol monoesters; (c) diglycerol monoaliphatic ethers of -branched C16-C24 alcohols, linear unsaturated C16-C22 alcohols, and linear saturated C12-C14 alcohols, and mixtures of these emulsifiers. See U.S. Pat. Nos. 5,287,207 and 5,500,451. Another emulsifier that may be used is polyglycerol succinate (PGS), which is formed from an alkyl succinate, glycerol, and triglycerol.

Such emulsifiers, and combinations thereof, may be added to the oil phase so that they constitute about 1% to about 20%, in certain examples about 2% to about 15%, and in certain other examples about 3% to about 12%, of the weight of the oil phase. In certain examples, coemulsifiers may also be used to provide additional control of cell size, cell size distribution, and emulsion stability, particularly at higher temperatures, for example greater than about 65° C. Examples of coemulsifiers include phosphatidyl cholines and phosphatidyl choline-containing compositions, aliphatic betaines, long chain C12-C22 dialiphatic quaternary ammonium salts, short chain C1-C4 dialiphatic quaternary ammonium salts, long chain C12-C22 dialkoyl(alkenoyl)-2-hydroxyethyl, short chain C1-C4 dialiphatic quaternary ammonium salts, long chain C12-C22 dialiphatic imidazolinium quaternary ammonium salts, short chain C1-C4 dialiphatic imidazolinium quaternary ammonium salts, long chain C12-C22 monoaliphatic benzyl quaternary ammonium salts, long chain C12-C22 dialkoyl(alkenoyl)-2-aminoethyl, short chain C1-C4 monoaliphatic benzyl quaternary ammonium salts, short chain C1-C4 monohydroxyaliphatic quaternary ammonium salts. In certain examples, ditallow dimethyl ammonium methyl sulfate (DTDMAMS) may be used as a coemulsifier.

Any photoinitiators included may be included at between about 0.05% and about 10%, and in some examples between about 0.2% and about 10% by weight of the oil phase. Lower amounts of photoinitiator allow light to better penetrate the HIPE foam, which can provide for polymerization deeper into the HIPE foam. However, if polymerization is performed in an oxygen-containing environment, it may be desired that there be enough photoinitiator present to initiate the polymerization and overcome oxygen inhibition. Photoinitiators can respond rapidly and efficiently to a light source with the production of radicals, cations, and other species that are capable of initiating a polymerization reaction. Photoinitiators selected for use in forming foams within contemplation of the present disclosure may absorb UV light at wavelengths of about 200 nanometers (nm) to about 800 nm, in certain examples about 250 nm to about 450 nm. If the photoinitiator is in the oil phase, suitable types of oil-soluble photoinitiators include benzyl ketals, α-hydroxyalkyl phenones, α-amino alkyl phenones, and acylphospine oxides. Examples of photoinitiators include 2,4,6-[trimethylbenzoyldiphosphine]oxide in combination with 2-hydroxy-2-methyl-1-phenylpropan-1-one (50:50 blend of the two is sold by Ciba Speciality Chemicals, Ludwigshafen, Germany as DAROCUR 4265); benzyl dimethyl ketal (sold by Ciba Geigy as IRGACURE 651); α-,α-dimethoxy-α-hydroxy acetophenone (sold by Ciba Speciality Chemicals as DAROCUR 1173); 2-methyl-1-[4-(methyl thio)phenyl]-2-morpholino-propan-1-one (sold by Ciba Speciality Chemicals as IRGACURE 907); 1-hydroxycyclohexyl-phenyl ketone (sold by Ciba Speciality Chemicals as IRGACURE 184); bis(2,4,6-trimethylbenzoyl)-phenylphosphineoxide (sold by Ciba Speciality Chemicals as IRGACURE 819); diethoxyacetophenone, and 4-(2-hydroxyethoxy)phenyl-(2-hydroxy-2-methylpropyl)ketone (sold by Ciba Speciality Chemicals as IRGACURE 2959); and Oligo [2-hydroxy-2-methyl-1-[4-(1-methylvinyl)phenyl]propanone](sold by Lamberti spa, Gallarate, Italy as ESACURE KIP EM.

The dispersed aqueous phase of a HIPE comprises water, and may also comprise one or more components, such as initiator, photoinitiator, or electrolyte, wherein in certain examples, the one or more components are at least partially water soluble.

One component included in the aqueous phase may be a water-soluble electrolyte. The water phase may contain from about 0.2% to about 40%, in certain examples from about 2% to about 20%, by weight of the aqueous phase of a water-soluble electrolyte. The electrolyte minimizes the tendency of monomers, comonomers, and crosslinkers that are primarily oil soluble to also dissolve in the aqueous phase. Examples of electrolytes include chlorides or sulfates of alkaline earth metals such as calcium or magnesium and chlorides or sulfates of alkali earth metals such as sodium. Such electrolyte can include a buffering agent for the control of pH during the polymerization, including such inorganic counterions as phosphate, borate, and carbonate, and mixtures thereof. Water soluble monomers may also be used in the aqueous phase, examples being acrylic acid and vinyl acetate.

Another component that may be included in the aqueous phase is a water-soluble free-radical initiator. The initiator can be present at up to about 20 mole percent based on the total moles of polymerizable monomers present in the oil phase. In certain examples, the initiator may be included in an amount of from about 0.001 to about 10 mole percent based on the total moles of polymerizable monomers in the oil phase. Suitable initiators include ammonium persulfate, sodium persulfate, potassium persulfate, 2,2′-azobis(N,N′-dimethyleneisobutyramidine)dihydrochloride, azo initiators, redox couples like persulfate-bisulfate, persulfate-ascorbic acid, and other suitable redox initiators. In certain examples, to reduce the potential for premature polymerization which may clog the emulsification system, addition of the initiator to the monomer phase may be performed near the end of the emulsification step, or shortly afterward.

Photoinitiator included in the aqueous phase may be at least partially water soluble, and may constitute between about 0.05% and about 10%, and in certain examples between about 0.2% and about 10%, by weight of the oil phase. Lower amounts of photoinitiator allow light to better penetrate the HIPE foam, which can provide for polymerization deeper into the HIPE foam. However, if polymerization is done in an oxygen-containing environment, there should be enough photoinitiator to initiate the polymerization and overcome oxygen inhibition. Photoinitiators can respond rapidly and efficiently to a light source with the production of radicals, cations, and other species that are capable of initiating a polymerization reaction. Photoinitiators selected for use to form foams within contemplation of the present disclosure may absorb UV light at wavelengths of from about 200 nanometers (nm) to about 800 nm, in certain examples from about 200 nm to about 350 nm, and in certain examples from about 350 nm to about 450 nm. If a photoinitiator is to be included in the aqueous phase, suitable types of water-soluble photoinitiators may include benzophenones, benzils, and thioxanthones. Examples of photoinitiators include 2,2′-Azobis[2-(2-imidazolin-2-yl)propane]dihydrochloride; 2,2′-Azobis[2-(2-imidazolin-2-yl)propane]disulfate dehydrate; 2,2′-Azobis(1-imino-1-pyrrolidino-2-ethylpropane)dihydrochloride; 2,2′-Azobis[2-methyl-N-(2-hydroxyethyl)propionamide]; 2,2′-Azobis(2-methylpropionamidine)dihydrochloride; 2,2′-dicarboxymethoxydibenzalacetone, 4,4′-dicarboxymethoxydibenzalacetone, 4,4′-dicarboxymethoxydibenzalcyclohexanone, 4-dimethylamino-4′-carboxymethoxydibenzalacetone; and 4,4′-disulphoxymethoxydibenzalacetone. Other suitable photoinitiators that can be used are listed in U.S. Pat. No. 4,824,765.

In addition to the previously described components other components may be included in either the aqueous or oil phase of a HIPE. Examples include antioxidants, for example hindered phenolics, hindered amine light stabilizers; plasticizers, for example dioctyl phthalate, dinonyl sebacate; flame retardants, for example halogenated hydrocarbons, phosphates, borates, inorganic salts such as antimony trioxide or ammonium phosphate or magnesium hydroxide; dyes and pigments; fluorescers; filler particles, for example starch, titanium dioxide, carbon black, or calcium carbonate; fibers; chain transfer agents; odor absorbers, for example activated carbon particulates; dissolved polymers; dissolved oligomers; and the like.

HIPE foam is produced from the polymerization of the monomers comprising the continuous oil phase of a HIPE. In certain examples, a HIPE foam layer may have one or more sublayers, and may be either homogeneous or heterogeneous polymeric open-celled foams. Homogeneity and heterogeneity relate to distinct layers within the same HIPE foam, which are similar in the case of homogeneous HIPE foams and differ in the case of heterogeneous HIPE foams. A heterogeneous HIPE foam may contain at least two distinct sublayers that differ with regard to their chemical composition, physical properties, or both; for example, sublayers may differ with regard to one or more of foam density, polymer composition, specific surface area, or pore size (also referred to as cell size). For example, for a HIPE foam if the difference relates to pore size, the average pore size in the respective sublayers may differ by at least about 20%, in certain examples by at least about 35%, and in still other examples by at least about 50%. In another example, if the differences in the sublayers of a HIPE foam layer relate to density, the densities of the layers may differ by at least about 20%, in certain examples by at least about 35%, and in still other examples by at least about 50%. For instance, if one layer of a HIPE foam has a density of 0.020 g/cc, another layer may have a density of at least about 0.024 g/cc or less than about 0.016 g/cc, in certain examples at least about 0.027 g/cc or less than about 0.013 g/cc, and in still other examples at least about 0.030 g/cc or less than about 0.010 g/cc. If the differences between the layers are related to the chemical composition of the HIPE or HIPE foam, the differences may reflect a relative amount difference in at least one monomer component, for example by at least about 20%, in certain examples by at least about 35%, and in still further examples by at least about 50%. For instance, if one sublayer of a HIPE or HIPE foam is composed of about 10% styrene in its formulation, another sublayer of the HIPE or HIPE foam may be composed of at least about 12%, and in certain examples of at least about 15%.

A HIPE foam layer structured to have distinct sublayers formed from differing HIPEs may provide a HIPE foam layer with a range of desired performance characteristics. For example, a HIPE foam layer comprising first and second foam sublayers, wherein the first foam sublayer has a relatively larger pore or cell size, than the second sublayer, when used in an absorbent article may more quickly absorb incoming fluids than the second sublayer. For example, when the HIPE foam layer is used to form an absorbent layer of a feminine hygiene pad, the first foam sublayer may be layered over the second foam sublayer having relatively smaller pore sizes, as compared to the first foam sublayer, which exert more capillary pressure and draw the acquired fluid from the first foam sublayer, restoring the first foam sublayer's ability to acquire more fluid from above. HIPE foam pore sizes may range from 1 to 200 m and in certain examples may be less than 100 m. HIPE foam layers of the present disclosure having two major parallel surfaces may be from about 0.5 to about 10 mm thick, and in certain examples from about 2 to about 10 mm. The desired thickness of a HIPE foam layer will depend on the materials used to form the HIPE foam layer, the speed at which a HIPE is deposited on a belt, and the intended use of the resulting HIPE foam layer.

The HIPE foam layers of the present disclosure are relatively open-celled. This refers to the individual cells or pores of the HIPE foam layer being in substantially unobstructed communication with adjoining cells. The cells in such substantially open-celled HIPE foam structures have intercellular openings or windows that are large enough to permit ready fluid transfer from one cell to another within the HIPE foam structure. For purpose of the present disclosure, a HIPE foam is considered “open-celled” if at least about 80% of the cells in the HIPE foam that are at least 1 m in size are in fluid communication with at least one adjoining cell.

In addition to being open-celled, in certain examples HIPE foams are adapted to be sufficiently hydrophilic to permit the HIPE foam to absorb aqueous fluids. In some examples the internal surfaces of a HIPE foam may be rendered hydrophilic by residual hydrophilizing surfactants or salts left in the HIPE foam following polymerization, by selected post-polymerization HIPE foam treatment procedures (as described hereafter), or combinations of both.

In certain examples, for example when it is used to form an absorbent layer of a feminine hygiene pad, a HIPE foam layer may be flexible and exhibit an appropriate glass transition temperature (Tg). The Tg represents the midpoint of the transition between the glassy and rubbery states of the polymer. In general, HIPE foams that have a Tg that is higher than the temperature of use can be strong but will also be relatively rigid and potentially prone to fracture (brittle). In certain examples, regions of the HIPE foams of the current disclosure which exhibit either a relatively high Tg or excessive brittleness will be discontinuous. Since these discontinuous regions will also generally exhibit high strength, they can be prepared at lower densities without compromising the overall strength of the HIPE foam.

HIPE foams intended for applications requiring flexibility should contain at least one continuous region having a Tg as low as possible, so long as the overall HIPE foam has acceptable strength at in-use temperatures. In certain examples, the Tg of this region will be less than about 40° C. for foams used at about ambient temperature conditions; in certain other examples Tg will be less than about 30° C. For HIPE foams used in applications wherein the use temperature is higher or lower than ambient temperature, the Tg of the continuous region may be no more than 10° C. greater than the use temperature, in certain examples the same as use temperature, and in further examples about 10° C. less than use temperature wherein flexibility is desired. Accordingly, monomers are selected as much as possible that provide corresponding polymers having lower Tg's.

HIPE foams useful for forming absorbent layers and/or sublayers within contemplation of the present disclosure, and methods for their manufacture, also include but are not necessarily limited to those foams and methods described in U.S. Pat. Nos. 10,752,710; 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; 5,500,451; 5,387,207; 5,352,711; U.S. Pat. Nos. 5,397,316; 5,331,015; 5,292,777; 5,268,224; 5,260,345; 5,250,576; 5,149,720; 5,147,345; and US 2005/0197414; US 2005/0197415; US 2011/0160326; US 2011/0159135; US 2011/0159206; US 2011/0160321; and US 2011/0160689, which are incorporated herein by reference to the extent not inconsistent herewith.

It will be appreciated that, when the absorbent foam layer material is cured or polymerized from a liquidous emulsion that has been deposited over a smooth, flat and level forming surface as described in references cited above, e.g., U.S. Pat. No. 10,752,710, because the emulsion is liquidous and of sufficiently low viscosity to enable it to substantially seek its own level over the forming surface, the resulting foam will have smooth and flat top and bottom surfaces following curing/polymerization, reflecting the smooth and flat forming surface on the bottom and the smooth and flat, self-leveled top surface of the precursor liquidous emulsion. The top/upper surface will typically exhibit Sdc and Sdr % values easily conforming to the ranges set forth in Table 1, above.

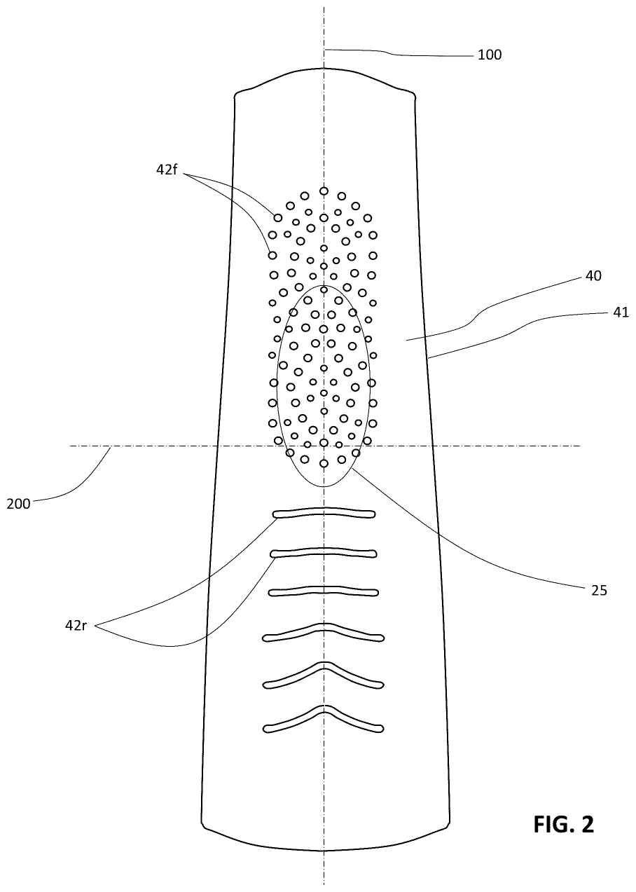

As reflected in FIGS. 1 and 2, the absorbent layer formed of HIPE foam may include one or more patterns of forward and rearward perforations 42f, 42r, including at least a first pattern disposed within an expected discharge location proximate the intersection of longitudinal and lateral axes 100, 200 of the pad. Perforations 42f, 42r may be punched, cut, molded, or otherwise formed through the entire z-direction depth of the HIPE foam absorbent layer, or only through a wearer-facing layer or partially into the wearer-facing portion thereof. When a HIPE foam absorbent layer is disposed in direct contact with a topsheet as described herein, with no intervening acquisition layer formed of another material, perforations 42f, 42r may serve as a group of “reservoirs” to receive, temporarily hold, and aid in distributing rapid discharges of relatively small quantities of menstrual fluid, until the HIPE foam has sufficient time to distribute and absorb the fluid via capillary action. Additionally, such perforations help decrease bending stiffness of the absorbent layer, which may help increase comfort of the pad for the wearer. A pattern of perforations having an average radius or other largest dimension of 1.0 mm to 4.0 mm, and more preferably 1.5 mm to 3.5 mm may be included, within, for example, the area occupied by a bonding region 25. The pattern may include perforations at a numerical density of 3.0 to 9.0 perforations per cm², and more preferably 4.0 to 8.0 perforations per cm². In selecting the appropriate average size, numerical density, and surface area occupied by the pattern of perforations, the manufacturer may wish to balance the volume of the “reservoirs” desired with the need to retain absorbent material in locations proximate to and about the expected discharge location. Additional details concerning configurations of such perforations in combination with examples of suitable absorbent layers may be found in U.S. Pat. No. 8,211,078.

Preferably, the topsheet 20 is bonded to the wearer-facing surface of the absorbent layer 40 in a manner that assures close proximity between the two, to provide for rapid fluid movement down through the topsheet to the absorbent layer 40, while not creating an unacceptable degree of occlusion (created, e.g., by overly large deposits of adhesive between these component) that would obstruct downward fluid movement. Bonding the topsheet to the absorbent layer also helps fix the absorbent layer 40 within the enveloped space, and helps unitize the overall structure of the pad, enhancing user/wearer impressions of quality. The topsheet 20 may be bonded to the absorbent layer 40 in any suitable manner, including that described in U.S. patent application Ser. No. 16/789,522.

### Bonding Between Topsheet and Absorbent Layer

In examples in which the topsheet material includes component fibers of sufficient hydrophilicity to cause it to wick, it may tend to retain fluid on its surfaces, and within the interstitial spaces between and along the surfaces of the fibers of the web material, unless there is sufficient direct contact maintained between the topsheet and the underlying absorbent layer to enable the fluid to move from fiber surfaces within the topsheet structure, directly to surfaces of material of the underlying absorbent layer. Prior to the time it is fully saturated, a nonwoven web material may not release retained fluid unless an adjacent material (with greater affinity for the fluid) is in sufficient direct contact. Accordingly, it is important to provide structure sufficient to maintain sufficient contact between the topsheet and the underlying absorbent layer, without obstructing fluid movement. No intervening layer or structure of material, or at least no intervening layer or structure of material less absorbent that that of the absorbent layer, should be interposed between the material of the topsheet 20 and the material of the absorbent layer 40, at least within the bonding region 25, more preferably over a majority of the wearer-facing surface area of the absorbent layer 40, and even more preferably over the entirety of the wearer-facing surface area of the absorbent layer 40—unlike systems provided in many current feminine hygiene pads, which include a distinct fluid acquisition/distribution material layer between the topsheet and the absorbent materials of the absorbent core.

In some examples, sufficient direct contact between the topsheet 20 and the absorbent layer 40 may be effected by deposit(s) of adhesive between the topsheet and the absorbent layer, adhesively bonding them in close z-direction proximity. The adhesive may be applied in a pattern or arrangement of adhesive deposits interspersed with areas in which no adhesive is present (unbonded areas), such that the adhesive holds the two layers in close z-direction proximity, while areas remain in which no adhesive is present to obstruct z-direction fluid movement between the layers.

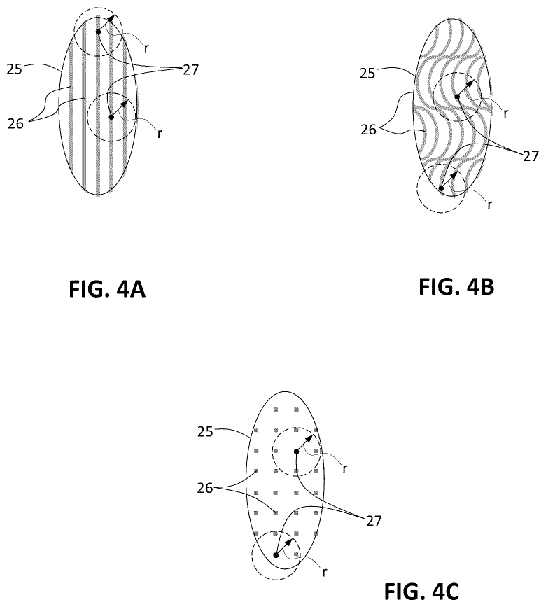

Referring to FIGS. 1 and 4A-4C, to ensure that the topsheet and absorbent layer are held in sufficiently close z-direction proximity at least in the area of the topsheet expected to receive a discharge of fluid, it may be desired to dispose a bonding region 25 on the pad at a location that includes the intersection of the longitudinal and lateral axes 100, 200. The bonding region 25 should be of sufficient size to be reliably present beneath the expected discharge location when the pad is in use, with reasonably minor variations of placement by the user/wearer within the underpants; accordingly, it may be desired that the bonding region have an area of at least 15 cm², more preferably at least 30 cm². Even more preferably, it may be desired that the bonding region have an area that is at least half of the total wearer-facing surface area of the absorbent layer. (Note: FIGS. 4A-4C are not presented herein as actual size or scale depictions.) To ensure that the topsheet 20 and the absorbent layer remain in sufficient z-direction proximity during use, it may be desired that, within any identifiable first point location 27 within the bonding region, at which the topsheet is bonded to the absorbent layer, there is a second point location at which the topsheet is bonded to the absorbent layer, within a 10 mm radius, more preferably within a 6 mm radius, 5 mm radius, 4 mm radius, and even more preferably within a 3 mm radius r of the first point location. Referring to FIGS. 3A-3C, depicting three non-limiting examples, it can be seen that a variety of patterns or arrangements of bonds (via adhesive deposits 26 or other bonding mechanisms) may be employed to impart this feature. Within radius r of each point location 27, there are a number of additional point locations where bonding between the topsheet and the absorbent layer is present in the examples depicted.

It will be appreciated that a continuous deposit of adhesive may be applied to bond the topsheet and the absorbent layer within the entirety of bonded region 25, but that such a continuous deposit of adhesive could form a barrier that would obstruct the movement of fluid from the topsheet to the absorbent layer. Accordingly, it is preferable that, in examples in which the bonding mechanism is deposits of adhesive, the deposits are disposed in a pattern or arrangement that is discontinuous or intermittent such that it creates bonded areas interspersed with unbonded areas between the topsheet and the absorbent layer. Additionally, when the absorbent layer is formed of an open-celled foam (such as a HIPE foam contemplated herein) it may be desired that the adhesive selected not effect adhesion to the absorbent layer via chemical, dispersive or diffusive adhesion with the foam layer at the adhesive deposit locations, but rather, that it effect adhesion to the foam layer mechanically, by flowing to a limited extent into the cells, at least partially assuming the shapes thereof, and solidifying in such position to form mechanical interlocks with the cell structures, which enable the adhesive to hold the topsheet to the absorbent layer. Such an adhesive may be preferred so as not to alter the molecular structure or composition of the foam material, potentially negatively affecting its fluid absorption properties or mechanical strength. In one example, a suitable adhesive for use with a HIPE foam may be H1750 hot melt adhesive from Bostik, Wauwatosa, Wisconsin (currently a subsidiary of Arkema, Columbes, France).

Unapertured topsheets for feminine hygiene pads formed of nonwoven web material and including or consisting predominately of hydrophilic fibers are known and have been included with some feminine hygiene products to date. (Herein, an “unapertured” nonwoven topsheet is one in which a majority of its surface area has not been subjected to any process that creates an arrangement of holes or apertures entirely therethrough, that persist prior to wetting of the topsheet, of an average size (greatest dimension) greater than 0.5 mm along an x-y planar direction.) Although favored by some consumers for their pleasant feel against the skin, topsheets formed of hydrophilic nonwoven web material have been disfavored by other consumers as a result of their substantial absorbency, i.e., capillary absorption and desorption pressures, causing them to resist drainage by conventionally included acquisition/distribution and absorbent layer structures. Following a discharge of menstrual fluid, a pad with such a topsheet overlying a conventional absorbent structure can feel to the user like a wet cloth held against the skin for an extended time period, which many users find objectionable. This dilemma has been present in the field for many years.

It has been discovered, however, that an unapertured fibrous nonwoven topsheet overlaid in direct, sufficient face-to-face proximate relationship with a HIPE foam absorbent layer or other layer adapted/manufactured to have capillary absorption capability sufficient to draw fluid from the topsheet, without any intervening less absorbent layers and in combination with other structural features as described herein, will be substantially drained of fluid by the absorbent layer, and regain a much drier feel against the skin following a discharge. It has been discovered that a suitably composed and manufactured HIPE foam absorbent layer as described herein, for example, has a greater affinity for menstrual fluid than such a topsheet, and thereby, has the capability to draw and retain fluid away from the topsheet when the two are disposed and held in sufficiently effective proximate, contacting relationship with each other. When the absorbent layer has a sufficient volume, it can serve this function over a reasonably suitable time of use of the pad.

A topsheet material having a relatively flat outward-facing (lower) surface as described above, may be more reliably bonded to a subjacent foam layer, via a discontinuous pattern of adhesive, than a topsheet material having a relatively more textured outward-facing surface. Thus, the combination of a topsheet material having the features described above, with a pattern of adhesive bonding as described above, provides synergy of features to enable more effective fluid acquisition, while providing a pleasing visibly-textured wearer-facing (upper) surface.

### Backsheet

The backsheet 30 may be positioned adjacent an outward-facing surface of the absorbent layer 40 and may be joined thereto by any suitable attachment methods. For example, the backsheet 30 may be secured to the absorbent layer 40 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent layer 40 is not joined directly to the backsheet 30.

The backsheet 30 may be impervious, or substantially impervious, to liquids (e.g., urine, menstrual fluid) and may be manufactured from a thin plastic film, although other flexible liquid impervious materials may also be used. As used herein, the term “flexible” refers to materials which are compliant and will readily conform to the general shape and contours of the human body. The backsheet 30 may prevent, or at least substantially inhibit, fluids absorbed and contained within the absorbent layer 40 from escaping and reaching articles of the wearer's clothing which may contact the pad 10 such as underpants and outer clothing. However, in some instances, the backsheet 30 may be made and/or adapted to permit vapor to escape from the absorbent layer 40 (i.e., the backsheet is made to be breathable), while in other instances the backsheet 30 may be made so as not to permit vapors to escape (i.e., it is made to be non-breathable). Thus, the backsheet 30 may comprise a polymeric film such as thermoplastic films of polyethylene or polypropylene. A suitable material for the backsheet 30 is a thermoplastic film having a thickness of from about 0.012 mm (0.5 mil) to about 0.051 mm (2.0 mils), for example. Any suitable backsheet known in the art may be utilized with the present invention.

Some suitable examples of backsheets are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523, 3,989,867, 3,156,242; WO 97/24097; U.S. Pat. Nos. 6,623,464; 6,664,439 and 6,436,508.

The backsheet may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489, 4,341,216, 4,713,068, 4,818,600; EP 203 821, EP 710 471; EP 710 472, and EP 0 793 952.

### Measurement Methods

### Surface Topography

In the Surface Topography measurement method the areal surface topology of a nonwoven web sample surface is measured using optical profilometry. The 3D surface data are then processed and analyzed to extract the microscale areal surface roughness parameter Sq (root mean square height), the surface complexity parameter Sdr (Developed Area), and the core height parameter Sdc. All sample preparation and testing is performed in a conditioned room maintained at 23±2° C. and 50±2% relative humidity, and prepared samples are kept in this environment for at least 24 hours prior to measurement.

### Sample Preparation

To prepare a sample of nonwoven web material to be obtained from finished feminine hygiene pad, the topsheet layer is removed from an absorbent article exposing the underlying absorbent layer. The topsheet layer is carefully removed in a manner that avoids distortion of the surface topography of the upper and lower surfaces of the material. A cryogenic spray (such as CYTO-FREEZE, Control Company, Houston Texas, or equivalent) may be used to facilitate clean separation of the topsheet material from the underlying absorbent layer and avoid tearing of the upper foam surface. Samples of topsheet materials with any tears or creases should not be used. Five replicate samples are prepared for testing.

### 3D Surface Image Acquisition

A three-dimensional (3D) surface topography image of the upper and lower surfaces of the topsheet sample and the upper surface of the absorbent layer of the sample pad is obtained using a DLP-based, structured-light 3D surface topography measurement system (a suitable surface topography measurement system is the MikroCAD Premium instrument commercially available from LMI Technologies Inc., Vancouver, Canada, or equivalent). The system includes the following main components: a) a Digital Light Processing (DLP) projector with direct digital controlled micro-mirrors; b) a CCD camera with at least a 1600×1200 pixel resolution; c) projection optics adapted to a measuring area of at least 140 mm×105 mm; d) recording optics adapted to a measuring area of 140 mm×105 mm; e) a table tripod based on a small hard stone plate; f) a blue LED light source; g) a measuring, control, and evaluation computer running surface texture analysis software (a suitable software is MikroCAD software with MountainsMap technology, or equivalent); and h) calibration plates for lateral (XY) and vertical (Z) calibration available from the vendor.

The optical 3D surface topography measurement system measures the surface height of a sample using the digital micro-mirror pattern fringe projection technique. The result of the measurement is a 3D data set of surface height (defined as the Z-axis) versus displacement in the horizontal (XY) plane. This 3D data set can also be thought of as an image in which every pixel in the image has an associated XY displacement, and the value of the pixel is the recorded Z-axis height value. The system has a field of view of 140×105 mm with an XY pixel resolution of approximately 85 microns, and a height resolution of 0.5 microns, with a total possible height range of 32 mm.

The instrument is calibrated according to manufacturer's specifications using the calibration plates for lateral (XY plane) and vertical (Z-axis) available from the vendor.

The sample is placed flat on the table beneath the camera. The sample may be very gently pulled taut (not to stretching) along X and Y dimensions to flatten out any large-scale waviness, and weights may be placed on the sample outside of the measurement area to hold it taut. A 3D surface topology image of the sample surface is collected by following the instrument manufacturer's recommended measurement procedures, which may include focusing the measurement system and performing a brightness adjustment. No pre-filtering options are used. The collected height image file is saved to the evaluation computer running the surface texture analysis software.

### 3D Surface Image Analysis

Analysis of a surface height image is initiated by opening the image in the surface texture analysis software. A recommended filtration process is described in ISO 25178-2:2021. Accordingly, the following filtering procedure is performed on each image: 1) If the sample surface is smaller than the image field of view, select the largest rectangular region of interest that can fit within the sample surface and crop the image to that size; 2) a Gaussian low pass S-filter with a nesting index (cut-off) of 2 mm to remove short scale components; 3) an F-operation of removing the least squares plane to level the surface; and 4) a Robust Gaussian high pass L-filter with a nesting index (cut-off) of 25 mm (ISO 16610-71) to remove long scale components. Both Gaussian filters are run utilizing end effect correction. This filtering procedure produces the S-L surface from which the areal surface texture parameters will be calculated.

This filtering procedure produces the surface from which the Sq (root mean square) values, as described in ISO 25178-2:2021, are calculated. Record the surface roughness values for Sq to the nearest 0.01 mm. This procedure is repeated for the remaining replicate samples. Average together the 5 replicate Sq measures and report these values to the nearest 0.01 mm.

The parameter Sdr, as described in ISO 25178-2:2021, is calculated on the filtered 3D topography image and recorded. Sdr is the ratio of the actual surface area of the sample to the projected horizontal area of the image and is given in percent (%). Record the surface complexity values for Sdr to the nearest 0.01%. This procedure is repeated for the remaining replicate samples. Average together the 5 replicate Sdr measures and report these values to the nearest 0.01%.

The core height value, Sdc, as described in ISO 25178-2:2021, is derived from the Areal Material Ratio (Abbott-Firestone) curve, which is the cumulative curve of the surface height distribution histogram versus the range of surface heights. The core height value is the height difference between the material ratios Smr1 and Smr2 as read off the Areal Material Ratio curve. Smr1, set to 2%, is the material ratio which separates the protruding peaks from the core roughness region. Smr2, set to 98%, is the material ratio which separates the deep valleys from the core roughness region. Record the surface height Sdc value to the nearest 0.01 mm. Average together the five replicate Sdc values and report to the nearest 0.01 mm.

### Caliper

The Caliper of a sample pad is measured as the distance between a reference platform on which the sample rests and a pressure foot that exerts a specified amount of pressure onto the sample over a specified amount of time. All measurements are performed in a laboratory maintained at 23° C.±2 C.° and 50%±2% relative humidity.

Caliper is measured with a manually-operated micrometer equipped with a pressure foot capable of exerting a steady pressure of 0.5 kPa±0.01 kPa onto the test sample. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter of 50 mm. The sample is supported by a horizontal flat reference platform that is larger than and parallel to the surface of the pressure foot. The system is calibrated and operated per the manufacturer's instructions.

The caliper of a sample pad is measured at a location centered at the intersection of its lateral and longitudinal axes, provided there are no set wrinkles or creases present at such location. If there are any set wrinkles or creases present at such location, adjust the measurement location such that the largest caliper portion of the pad having no set wrinkles or creases is disposed beneath the pressure foot.

Sample pads are conditioned at 23° C.±2 C.° and 50%±2% relative humidity for 2 hours prior to testing. To measure caliper, first zero the micrometer against the horizontal flat reference platform. Place the sample pad on the platform with the desired measurement location centered below the pressure foot. Gently lower the pressure foot with a descent rate of 1.0 mm±0.1 mm per second until the full pressure is exerted on the sample. Wait 5 seconds and then record the caliper of the sample to the nearest 0.01 mm. In like fashion, repeat for a total of 10 replicate samples. Calculate the arithmetic mean for the Caliper and report to the nearest 0.01 mm.

In view of the foregoing description, the following non-limiting examples are contemplated:

1. An absorbent article having a liquid permeable topsheet (20), a liquid impermeable backsheet (30), and an absorbent layer (40) enveloped therebetween, wherein:
the topsheet (20) comprises a fibrous nonwoven material and has an upper topsheet surface and a lower topsheet surface, wherein:
   the lower topsheet surface exhibits a lower topsheet surface Sdc less than 0.350 mm, more preferably less than 0.300 mm, and
   the upper topsheet surface exhibits an upper topsheet surface Sdc that is greater than the lower topsheet surface Sdc; and
the absorbent layer (40) comprises a layer of open-cell foam having an upper foam surface that is in direct facing contact with the lower topsheet surface.

2. The absorbent article of example 1, wherein the upper topsheet surface exhibits an upper topsheet surface Sdc that is at least 15 percent greater, preferably at least 20 percent greater, than the lower topsheet surface Sdc.

3. The absorbent article of either of the preceding examples wherein the upper topsheet surface exhibits an upper topsheet surface Sdc that is greater than 0.360 mm, more preferably greater than 0.420 mm.

4. An absorbent article having a liquid permeable topsheet (20), a liquid impermeable backsheet (30), and an absorbent layer (40) enveloped therebetween, wherein:
the topsheet comprises a fibrous nonwoven material and has an upper topsheet surface and a lower topsheet surface, wherein:
   the lower topsheet surface exhibits a lower topsheet surface Sdr % less than 0.80, more preferably less than 0.750; and
   the upper topsheet surface exhibits an upper topsheet surface Sdr % that is greater than the lower topsheet surface Sdr %; and
the absorbent layer (40) comprises a layer of open-cell foam having an upper foam surface that is in direct facing contact with the lower topsheet surface.

5. The absorbent article of example 4 wherein the upper topsheet surface exhibits an upper topsheet surface Sdr % that is at least 30 percent greater, preferably at least 35 percent greater, than the lower topsheet surface Sdr %.

6. The absorbent article of either of examples 4 or 5 wherein the upper topsheet surface exhibits an upper topsheet surface Sdr % that is greater than 1.0, more preferably greater than 1.1.

7. The absorbent article of any of the preceding examples wherein the upper foam surface has an upper foam surface Sdc, and the lower topsheet surface Sdc is less than 250 percent, more preferably less than 200 percent, of the upper foam surface Sdc.

8. The absorbent article of any of the preceding examples wherein the upper foam surface has an upper foam surface Sdr % and the lower topsheet surface Sdr % that is less than 5,000 percent, more preferably less than 3,500 percent, of the upper foam surface Sdr %.

9. The absorbent article of any of the preceding examples wherein the topsheet (20) comprises a fibrous spunbond nonwoven material.

10. The absorbent article of example 9, wherein the spunbond nonwoven material comprises an ordered, patterned arrangement of alternating built-up regions and attenuated regions.

11. The absorbent article of any of examples 1-8 wherein the topsheet (20) comprises a carded staple fiber nonwoven web material.

12. The absorbent article of any of the preceding examples wherein the topsheet (20) comprises bicomponent fibers.

13. The absorbent article of example 12 wherein the bicomponent fibers have a sheath/core configuration.

14. The absorbent article of either of examples 12 or 13 wherein the bicomponent fibers have an exposed component comprising polyethylene.

15. The absorbent article of example 14 comprising fusion bonds between bicomponent fibers, wherein the bonds are formed by heating without compression.

16. The absorbent article of any of the preceding examples wherein the open-cell foam comprises one or more of polyurethane foam and HIPE foam, preferably HIPE foam.

17. The absorbent article of any of the preceding examples wherein the layer of open-cell foam has upper and lower sublayers having upper and lower sublayer average cell sizes, wherein the upper sublayer average cell size is greater than the lower sublayer average cell size.

18. The absorbent article of any of the preceding examples wherein the upper foam surface Sdc is no greater than 0.20 mm, preferably no greater than 0.15 mm.

19. The absorbent article of any of the preceding examples wherein the upper foam surface Sdr % is no greater than 0.040%, preferably no greater than 0.030%.

20. The absorbent article of any of the preceding examples comprising a central bonding region (25) straddling the an intersection of longitudinal (100) and lateral (200) axes, the central bonding region having a size of at least 15 cm2, more preferably at least 30 cm2, within which the topsheet (20) is bonded to the absorbent layer (40) via a discontinuous pattern of adhesive bonds (26), and within which, within any identifiable first point location (27) of an adhesive bond (26) within the bonding region (25), at which the topsheet is bonded to the absorbent layer, there is a second point location at which the topsheet is bonded to the absorbent layer by adhesive, within a 10 mm radius, more preferably within a 6 mm radius, 5 mm radius, 4 mm radius, and even more preferably within a 3 mm radius r of the first point location.

21. The absorbent article of any of the preceding examples wherein the topsheet (20) comprises hydrophilic fibers, or fibers that have been surface-treated with a surfactant.

22. The absorbent article of example 13 wherein the topsheet lower surface has been surface-treated with a surfactant.

23. The absorbent article of either of examples 21 or 22 wherein the topsheet (20) comprises hydrophobic fibers.

24. The absorbent article of any of the preceding examples, having a caliper no greater than 7 mm, preferably no greater than 6 mm, more preferably no greater than 5 mm, even more preferably no greater than 4 mm, and still more preferably no greater than 3.5 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety to the extent not inconsistent herewith and unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article having a liquid permeable topsheet, a liquid impermeable backsheet, and an absorbent layer enveloped therebetween, wherein:
the topsheet comprises a fibrous nonwoven material and has an upper topsheet surface and a lower topsheet surface, wherein:
the lower topsheet surface exhibits a lower topsheet surface Sdc less than 0.350 mm, and
the upper topsheet surface exhibits an upper topsheet surface Sdc that is greater than the lower topsheet surface Sdc; and
the absorbent layer comprises a layer of open-cell foam having an upper foam surface that is in direct facing contact with the lower topsheet surface.

2. The absorbent article of claim 1, wherein the upper topsheet surface exhibits an upper topsheet surface Sdc that is at least 15 percent than the lower topsheet surface Sdc.

3. The absorbent article of claim 1 wherein the upper topsheet surface exhibits an upper topsheet surface Sdc that is greater than 0.360 mm.

4. The absorbent article of claim 1 wherein the upper foam surface has an upper foam surface Sdc, and the lower topsheet surface Sdc is less than 250 percent of the upper foam surface Sdc.

5. The absorbent article of claim 1 wherein the upper foam surface has an upper foam surface Sdr % and the lower topsheet surface Sdr % that is less than 5,000 percent of the upper foam surface Sdr %.

6. The absorbent article of claim 1 wherein the topsheet comprises a fibrous spunbond nonwoven material comprising an ordered, patterned arrangement of alternating built-up regions and attenuated regions.

7. The absorbent article of claim 1 wherein the topsheet comprises a carded staple fiber nonwoven web material comprising bicomponent fibers having a sheath/core configuration.

8. The absorbent article of claim 1 wherein the open-cell foam comprises one or more of polyurethane foam and HIPE foam, wherein the layer of open-cell foam has upper and lower sublayers having upper and lower sublayer average cell sizes, wherein the upper sublayer average cell size is greater than the lower sublayer average cell size.

9. The absorbent article of claim 1 comprising a central bonding region straddling the an intersection of longitudinal and lateral axes, the central bonding region having a size of at least 15 cm2, within which the topsheet is bonded to the absorbent layer via a discontinuous pattern of adhesive bonds, and within which, within any identifiable first point location of an adhesive bond within the bonding region, at which the topsheet is bonded to the absorbent layer, there is a second point location at which the topsheet is bonded to the absorbent layer by adhesive, within a 10 mm radius r of the first point location.

10. The absorbent article of claim 1 wherein the topsheet comprises hydrophilic fibers, or fibers that have been surface-treated with a surfactant.

11. An absorbent article having a liquid permeable topsheet, a liquid impermeable backsheet, and an absorbent layer enveloped therebetween, wherein:
the topsheet comprises a fibrous nonwoven material and has an upper topsheet surface and a lower topsheet surface, wherein:
the lower topsheet surface exhibits a lower topsheet surface Sdr % less than 0.80; and
the upper topsheet surface exhibits an upper topsheet surface Sdr % that is greater than the lower topsheet surface Sdr %; and
the absorbent layer comprises a layer of open-cell foam having an upper foam surface that is in direct facing contact with the lower topsheet surface.

12. The absorbent article of claim 11 wherein the upper topsheet surface exhibits an upper topsheet surface Sdr % that is at least 30 percent greater than the lower topsheet surface Sdr %.

13. The absorbent article of claim 11 wherein the upper topsheet surface exhibits an upper topsheet surface Sdr % that is greater than 1.0.

14. The absorbent article of claim 11 wherein the upper foam surface has an upper foam surface Sdc, and the lower topsheet surface Sdc is less than 250 percent of the upper foam surface Sdc.

15. The absorbent article of claim 11 wherein the upper foam surface has an upper foam surface Sdr % and the lower topsheet surface Sdr % that is less than 5,000 percent of the upper foam surface Sdr %.

16. The absorbent article of claim 11 wherein the topsheet comprises a fibrous spunbond nonwoven material comprising an ordered, patterned arrangement of alternating built-up regions and attenuated regions.

17. The absorbent article of claim 11 wherein the topsheet comprises a carded staple fiber nonwoven web material comprising bicomponent fibers having a sheath/core configuration.

18. The absorbent article of claim 11 wherein the open-cell foam comprises one or more of polyurethane foam and HIPE foam wherein the layer of open-cell foam has upper and lower sublayers having upper and lower sublayer average cell sizes, wherein the upper sublayer average cell size is greater than the lower sublayer average cell size.

19. The absorbent article of claim 11 comprising a central bonding region straddling the an intersection of longitudinal and lateral axes, the central bonding region having a size of at least 15 cm2, within which the topsheet is bonded to the absorbent layer via a discontinuous pattern of adhesive bonds, and within which, within any identifiable first point location of an adhesive bond within the bonding region, at which the topsheet is bonded to the absorbent layer, there is a second point location at which the topsheet is bonded to the absorbent layer by adhesive, within a 10 mm radius r of the first point location.

20. The absorbent article of claim 11 wherein the topsheet comprises hydrophilic fibers, or fibers that have been surface-treated with a surfactant.
